(19) United States
(12) Patent Application Publication
(10) Pub. No.: US 2023/0393042 A1
(43) Pub. Date: Dec. 7, 2023

(54) PRETREATMENT METHOD, PRESERVATION METHOD, AUTOMATIC TREATMENT SYSTEM AND DETECTION METHOD FOR URINE SAMPLE

(71) Applicants: JIANGSU QLIFE MEDICAL TECHNOLOGY GROUP CO., LTD., Nanjing, Jiangsu (CN); NANJING QLIFE MEDICAL TECHNOLOGY CO., LTD., Nanjing, Jiangsu (CN)

(72) Inventors: Xiaoliang CHENG, Nanjing (CN); Jing YU, Nanjing (CN); Yue ZHOU, Nanjing (CN); Wei ZHANG, Nanjing (CN); Kejia ZHENG, Nanjing (CN)

(73) Assignees: JIANGSU QLIFE MEDICAL TECHNOLOGY GROUP CO., LTD., Nanjing, Jiangsu (CN); NANJING QLIFE MEDICAL TECHNOLOGY CO., LTD., Nanjing, Jiangsu (CN)

(21) Appl. No.: 18/036,646

(22) PCT Filed: Aug. 17, 2022
(86) PCT No.: PCT/CN2022/112947

§ 371 Date: Jul. 20, 2023

(30) Foreign Application Priority Data
Jan. 17, 2022 (CN) 202210050595.6

(51) Int. Cl. G01N 1/42; G01N 1/34; G01N 30/72; G01N 30/34; C12N 9/76; C12N 9/06

(52) U.S. Cl. CPC G01N 1/42; G01N 1/34; G01N 30/72; G01N 30/34; G01N 2001/4088; C12N 9/0032; C12Y 304/21004; C12Y 105/03004; C12N 9/6427

(57) ABSTRACT

The invention discloses a pretreatment method, a preservation method, an automatic treatment system and a detection method for a urine sample, and directs to the technical field of biological detection. The pretreatment method comprises subjecting a urine sample after protein lysis to a reductive alkylation treatment, followed by protein enrichment and enzymolysis. The protein enrichment is performed on the sample after the reductive alkylation treatment using a PVDF filter plate for protein enrichment; The invention also provides an automatic treatment system and an automatic sample treatment method. The treatment system greatly reduces the labor intensity of people, is beneficial to facilitate the treatment efficiency of urine sample treatment, meets the requirements of high-flux and automated pretreatment of the proteomics, and accommodates the reproducibility and flux of current clinical needs.

### FIELD OF THE PRESENT DISCLOSURE

The present application claims the priority of the Chinese patent application with the application number of 202210050595.6 and the title of the invention of “Pretreatment Method, Preservation Method, Automatic Treatment system and Detection Method for Urine Sample” filed on Jan. 17, 2022 in the China National Intellectual Property Administration, the entire contents of which are incorporated herein by reference.

### TECHNICAL FIELD

The invention relates to the technical field of biological detection and, in particular, to a pretreatment method, a preservation method, an automatic treatment system and a detection method for a urine sample.

### BACKGROUND ART

Changes in the type and amount of proteins in urine carry various information about the occurrence, development and prognosis of urinary diseases. Urine proteomics is one of the most effective methods to interpret the information contained in the urine proteins. In the field of clinical application, a multi-center and large samples are often required for research analysis so as to obtain more accurate results. However, due to the large volume of urine samples, big difference in protein concentration and the presence of bacteria and other microbial characteristics, how to optimize the preservation of clinical urine samples is one of the urgent problems to be solved recently.

Urine samples have been widely used in scientific research fields because of their advantages in respect of simple, non-invasive and rapid collection. However, the urine samples are also faced with problems such as how to preserve the samples for a longer time and how to simplify transportation thereof. If the quality control of any step thereof is not effective, the reliability of protein detection results may be influenced. Under conventional conditions, the urine samples are collected clinically and then detected, or stored in the sample bank in the form of the urine tube. but this is not advantageous for storage since it will occupy a large volume of the refrigerator. In addition, there are many interfering compounds in urine, such as uric acid, creatinine, ammonia and other non-protein nitrogen compounds, sulfate and so on, resulting in dramatic changes in urine pH, which will accelerate the decay of urine. At the same time, there will be bacteria in urine, and the bacteria still have activity in long-term storage, resulting in the possibility of protein in urine being decomposed by the bacteria, which affects the reliability of final results. However, it has been proposed by the researchers that the urine protein is preserved on the membrane, which is convenient for transportation, less occupancy, and can be preserved for at least a half of year. However, the area of the membrane material used here is large, and is about 40 mm. Further, the operation of preserving urine protein can only be performed on a single sample, and high-flux treatment cannot be available, which limits the clinical use of membrane material for preserving urine protein.

Proteomics is a science that studies the composition and variation rule of protein in urine, serum or organism by taking the proteome as an object for research. In 1997, HEINE et al. identified 34 proteins by the high-performance liquid chromatography-electrospray ionization-MS (HPLC-ESI-MS) and obtained urine protein fragment maps of normal people. Subsequently, LEE et al. identified 600 protein molecules in urine by the mass spectrometry and expanded the urine proteome database. Subsequently, ADACHI et al. identified 1543 urine proteins by the linear ion trap-orbitrap (LTQ-Orbitrap) technique. Alex et al. identified 2362 protein molecules by applying two-dimensional gel electrophoresis (2-DE)+LTQ-Orbitrap & LC-MS/MS to make a more comprehensive analysis on the urine protein components of normal people.

Over the past two decades, the mass spectrometry (MS)-based methods have become the preferred method for quantification of proteins in biological samples with high confidence and depth coverage, and have greatly facilitated the annotation of signal transduction networks within organisms, elucidating the interactions of proteins in different physiopathological states, improving the diagnosis of disease mechanisms and molecular comprehension. Typical proteomics experimental process mainly includes protein lysis (extracting the protein from the sample), protein content determination (determining the protein content in the solution), reduction/alkylation (breaking disulfide bonds such that the protein molecules changes their form from a sphere to a chain as much as possible and increase the solubility of protein, and then the alkylation reagent binds to the free sulfhydryl of the protein, with exposing as many digestion sites as possible), proteolysis (trypsin digests the protein sample into multiple peptide fragments), desalting (removing the inorganic salt components presented in the peptide fragment solution and enriching, concentrating and lyophilizing the peptide fragments), and final analysis by the chromatography and mass spectrometry.

Since entering the era of precision medicine, the development of proteomics is developing towards the clinical application of precision medicine. Therefore, how to rapidly improve the detection flux has become one of the important research areas in the current omics. Among them, the protein is extracted from clinical samples to obtain a protein solution for quantitative determination, reductive alkylation, proteolysis, peptide fragment desalting and enrichment. The above process all belongs to the traditional manual sample treatment stage in the proteomics. The traditional manual pretreatment experimental process is slow, has too many steps and is laborious, with total time of 8-18 hours etc. for the whole process. Generally, the experimenter cannot complete the pretreatment process for a batch of samples within one working day, and also can hardly provide the reproducibility and flux to meet the current clinical needs. Thus, the high-flux and automated pretreatment process of the proteomics has become one of the innovative technologies urgently needed in the whole industry.

In view of this, the invention has been proposed.

### SUMMARY OF THE PRESENT DISCLOSURE

It is an object of the invention to provide a urine sample pretreatment method, preservation method, automated treatment system and detection method to solve the above-mentioned technical problem.

The quality of the whole project is determined by the preservation of clinical samples. The sample pretreatment process is a crucial step in the whole proteomics analysis process, which determines the sensitivity and accuracy of the whole sample analysis. Therefore, the invention aims to find a more efficient urine sample preservation method and a high-flux automated sample protein pretreatment method, so as to improve the quality of the whole project while reducing the protein loss during the pretreatment process, improve the reproducibility and stability of the experiment, and improve the performance and flux of the whole experiment process.

The invention is carried out as follows.

The invention provides a preservation method for a urine sample, comprising: subjecting the urine sample after protein lysis to a reductive alkylation treatment, and then to a protein enrichment;
wherein the protein enrichment is performed on the sample after the reductive alkylation treatment using a PVDF filter plate for protein enrichment;
the volume ratio of a lysate used for protein lysis to the urine sample to be lysed is 1:0.1-9.

The present inventors have found that, unlike the physical protein entrapment effect of the FASP membrane, the treatment method provided by the invention uses the PVDF membrane to adsorb proteins onto the membrane surface. In theory, each well of the PVDF membrane in the PVDF filter plate can adsorb 20-25 μg of proteins (different size filter plates or filter tubes can correspond to different amounts of protein adsorption). Therefore, the proteins in the urine sample can be retained on the PVDF filter plate, and the urine proteins can be stored in solid form. The inventors demonstrate through experiment that the method of selecting the PVDF filter plate for urine protein preservation is relatively reliable, and can ensure stable urine protein quantity and quality within 1 year. The PVDF filter plate described above may be selected from the commercially available MultiScreen HTS IP. The manufacturer thereof is Millipore, and the model is MSIPS451.

In a preferred embodiment of the invention, the above-mentioned lysate is at least one selected from the group consisting of urea, thiourea, guanidine hydrochloride, tris (hydroxymethyl) aminomethane-hydrochloride, phenylmethylsulfonyl fluoride, sodium dodecyl sulfate, sodium deoxycholate and 3-[3-(cholamidopropyl) dimethylammonio]-1-propanesulfonate (CHAPS, CAS No. 75621-03-3). For example, it is a combination of urea and CHAPS.

In an optional embodiment, the lysate is selected from urea and the final concentration of the urea in the urine sample to be lysed is 1M-5M, for example, 1M, 1.5M, 2M, 2.5M, 3M, 3.5M, 4M, 4.5M or 5M.

The inventors have found that the lysate acts as a denaturant to denature the protein, refolding and facilitating solubilization of the protein. However, too high concentration of lysate results in a decrease in the affinity of the protein to positively bind to the PVDF membrane, resulting in a decrease in the amount of protein adsorbed on the membrane. When the concentration of urea is higher than a certain concentration, the performance of PVDF filter plate may also be affected. The inventors demonstrate by experimental verification that it results in the effect of protein adsorption efficiency of PVDF membrane material if the concentration of protein lysate is too high, while only little or substantially no protein is adsorbed on PVDF membrane, thereby resulting in that no protein can be detected when it is on line subsequently.

In a preferred embodiment of the application of the invention, the PVDF membrane material has an overall optimal efficiency for protein adsorption when the final concentration of urea in the urine sample to be lysed is 3M.

In an optional embodiment, the diluent for lysate is at least one selected from the group consisting of ammonium bicarbonate, tris (hydroxymethyl) aminomethane-hydrochloride solution (Tris-HCl), phosphate solution (PBS).

In a preferred embodiment of the invention, the method further comprises, prior to protein enrichment, activating the PVDF filter plate, equilibrating with the lysate after the activation, and then transferring the sample after the reductive alkylation treatment to the equilibrated PVDF filter plate for protein enrichment.

The residual chemicals in the PVDF membrane itself can be removed by activation, so as to avoid the interference of residual chemicals in the PVDF membrane itself on the adsorption and detection of urine protein.

In an optional embodiment, an activating agent used for the activation is an alcohol species. Alcohol species can change the PVDF membrane from a hydrophobic state to a hydrophilic state, and simultaneously activate the positive groups on the PVDF membrane, making them easier to bind to negatively charged proteins, thereby improving the quantity and quality of peptide and protein detection in the urine samples. In other embodiments, this includes, but is not limited to, activation of PVDF filter plates by an activating agent based on the theory of similarity and intermiscibility.

The activating agent used for activation may be at least one selected from the group consisting of methanol, ethanol, acetonitrile and isopropanol. The reagent for equilibration may be a protein lysate.

The above-mentioned reductive alkylation includes two steps: i.e., reduction and alkylation, which can be performed with reference to the existing process of reductive alkylation, without being limited. In some embodiments, the reagent used in the reductive alkylation may be selected from at least one of dithiothreitol, iodoacetamide, chloroacetamide and tris (2-carboxyethyl) phosphine hydrochloride.

The above-mentioned PVDF filter plate after protein enrichment can be directly used for urine protein preservation, for example, directly stored at −80° C. to −20° C.

In a preferred embodiment of the invention, it is preserved at −80° C. for urinary protein preservation through the PVDF filter plates after protein enrichment with best preservation effect.

The invention also provides a pretreatment method for a urine sample comprising: subjecting the urine sample after protein lysis to a reductive alkylation treatment, then to a protein enrichment, performing enzymolysis after the enrichment, and concentrating and lyophilizing the resultant.

The protein enrichment is performed on the sample after the reductive alkylation treatment using a PVDF filter plate for protein enrichment.

The volume ratio of a lysate used for protein lysis to the urine sample to be lysed is 1:0.1-9.

The PVDF filter plates include, but are not limited to 96-well plates or 384-well plates containing PVDF filtration membranes. Note that the 96-well plate or 384-well plate herein refers to a filter plate having 96 filter tubes or 384 filter tubes, except that the structure of the filter tubes is similar to that of the “wells” in a conventional 96-well plate or 384-well plate.

The pretreatment method provided by the invention controls the whole urine pretreatment process period within 4 hours, and can perform pretreatment of 96 or more flux samples at the same time, with great technical advantage of high treatment efficiency. Specifically, since PVDF membrane of each well can adsorb 20-μg of protein, the step of protein quantitative detection in the subsequent pretreatment process can be omitted. Meanwhile, after protein adsorption using the PVDF filter plate, multiple washing and centrifugation operations are added in the protein enrichment step, so that the interfering substances (such as inorganic salts, urine sediment, cells, bacteria and debris) originally present in the protein sample are removed, which has played a desalting effect. Therefore, compared with traditional proteomics experimental process, the present application omits protein quantification and desalting, which significantly reduces sample pretreatment time and reduces costs. Further, the pretreatment method of the present application uses a PVDF membrane of a multiwell plate, which can treat 96 or more urine samples at once, thereby overcoming the flux limitation of FASP. Therefore, it provides a high-flux urine sample pretreatment method which can be applied to automatic proteomics analysis applications of urine samples.

Such urine samples include, but are not limited to animal (without human) urine samples and ex vivo urine samples.

In a preferred embodiment of the invention, it further comprises collecting a filtrate from the PVDF filter plate subsequent to the enzymolysis.

In an optional embodiment, the enzymes employed for the enzymolysis are trypsin and lysinase (LysC). The enzymolysis is performed under shaking conditions. In an optional embodiment, the enzymolysis time is 1-18 h.

Alternatively, the protein is divided into a plurality of small peptide fragments after enzymolysis by mixing the sample protein to the protease (Trypsin, lysinase (LysC)) in a ratio of 1:10-100 based on the mass ratio.

In an optional embodiment, the protein enrichment comprises applying the sample after the reductive alkylation treatment to the PVDF filter plate and after centrifugation, washing the centrifuged sample with an eluent. In particular, disulfide bonds in the protein structure are broken by the reductive alkylation to bind to free sulfhydryl of the protein, thereby exposing as many digestion sites as possible for subsequent digestion applications.

In a preferred embodiment of the use of the invention, the above-mentioned lysate is at least one selected from the group consisting of urea, thiourea, guanidine hydrochloride, tris (hydroxymethyl) aminomethane-hydrochloride, phenylmethylsulfonyl fluoride, sodium dodecyl sulfate, sodium deoxycholate and 3-[3-(cholamidopropyl) dimethylammonio]-1-propanesulfonate. The CAS number for 3-[3-(cholamidopropyl) dimethylammonio]-1-propanesulfonate is 75621-03-3, abbreviated as CHAPS.

In an optional embodiment, the lysate is selected from urea and the final concentration of the urea in the urine sample to be lysed is 1M-5M,

The inventors find that when the traditional FASP method is used for urine sample pretreatment, the protein lysate used is 8M urea. However, when the 8M urea is used to lyse the protein in urine and then contacts with PVDF filter plate, the affinity of positively binding protein to the PVDF membrane material is seriously decreased, resulting in a serious decrease in the efficiency of membrane adsorption of protein, even without binding to the protein. Therefore, the concentration of urea solvent must be decreased to enable the PVDF filter plate to perform the function of protein adsorption. Therefore, according to the invention, the PVDF filter plate is applied to the FASP pretreatment method according to the characteristics of the PVDF material that has strict requirement for the concentration of urea solvent, and the advantages of the FASP method in removing impurities existing in the protein pretreatment process and the smaller solution volume of the whole system, and the concentration of urea solvent is optimized at the same time. A rapid and clean urine proteomic pretreatment method is thus developed.

The invention also provides an automatic treatment system for an urine sample, comprising a urine sample storage unit, a treating fluid supply unit, a PVDF filter plate supply unit, a sample suction unit, a protein collection unit and an enzyme storage unit, wherein the urine sample storage unit, the treating fluid supply unit, the PVDF filter plate supply unit, the sample suction unit, the protein collection unit and the enzyme storage unit are electrically connected to a control terminal for automatic control.

In an optional embodiment, the control terminal is a computer. Such sample suction units include, but are not limited to, multichannel pipettes and the like.

In a preferred embodiment of the invention, the treatment system further comprises a lysis reaction vessel supply unit, a shaker, a concentrator and a PCR plate.

The treating fluid supply unit includes a lysate supply unit, a reducing agent supply unit, an alkylating agent supply unit, an alkylation reaction terminating agent supply unit, an eluent supply unit, an activating agent supply unit, and a reconstitution solvent supply unit. The treating fluid supply unit may be a twelve-channel tank, with a different reagent supply unit provided in each channel.

The invention also provides a treatment method for a urine sample by applying the automatic treatment system for the urine sample, comprising:
(1) protein lysis: taking a urine sample to be tested from a urine sample storage unit into a lysis reaction vessel by using a sample suction unit, and sucking a lysate from a treating fluid supply unit into the lysis reaction vessel via the sample suction unit to perform the protein lysis;
(2) reductive alkylation: sucking a reducing agent from the treating fluid supply unit into the lysis reaction vessel via the sample suction unit to perform a reduction reaction, sucking an alkylating agent from the treating fluid supply unit into the lysis reaction vessel via the sample suction unit to perform an alkylation reaction, and after the reaction, sucking an alkylation reaction terminating agent from the treating fluid supply unit via the sample suction unit into the lysis reaction vessel so as to terminate the alkylation reaction;
(3) protein enrichment: activating the PVDF filter plate by sucking an activating agent from the treating fluid supply unit into the PVDF filter plate via the sample suction unit, equilibrating the PVDF filter plate by sucking the lysate from the treating fluid supply unit into the PVDF filter plate via the sample suction unit, then adding a product of the reductive alkylation treatment to the PVDF filter plate via the sample suction unit, and centrifuging the same;
(4) proteolysis: sucking an enzyme reaction solution from an enzyme storage unit into the PVDF filter plate via the sample suction unit to perform an enzymolysis reaction, then sucking an eluent from the treating fluid supply unit into the PVDF filter plate via the sample suction unit to elute an enzymolysis reaction product, and then combining the eluent; and
(5) concentrating and lyophilizing: concentrating and lyophilizing the eluent.

In an optional embodiment, the protein lysis is performed on a thermostatic mixing shaker with vortex mixing at a speed of 1000 rpm.

In an optional embodiment, the reduction reaction, the alkylation reaction, and the termination of the alkylation reaction are all performed under vortex conditions at a rotational speed of 1000 rpm, such as 20 min at room temperature during the reduction reaction, 20 min at room temperature during the alkylation reaction, and 1 min at room temperature during the termination of the alkylation reaction.

The invention also provides a method of mass spectrometric detection for a urine sample, which is directed for the purpose of non-diagnosis of disease, comprising: treating the urine sample by using the method above, and then detecting peptide fragment by using a mass spectrometer;
setting a mobile phase A as an aqueous solution containing 0.05-0.2% formic acid and a mobile phase B as 80% acetonitrile containing 0.05-0.2% formic acid for gradient elution, with a flow rate of 200-300 nl/min and a column temperature of 30-55° C.;
preferably, the gradient elution has procedures of 1-6 min, 1%-8% B, 6-30 min, 8-99% B;
setting mass spectrometry parameters, including a mass spectrum full scan resolution of 240,000, 120,000, 70,000, 60,000, 45,000, 30,000, 17,500, or 7,500@m/z 200, AGC of 1E5-3E6, maximum ion sample injection time of ms, a scan range of m/z 200-2000, normalized collision energy of 15-27%; a secondary mass spectrum scan resolution of 240,000, 120,000, 70,000, 60,000, 45,000, 30,000, 17,500, 15,000 or 7,500@m/z 200, a scanning range of m/z 200-2000, an AGC of 1E5-1E6, maximum ion injection time of 10-100 ms, dynamic exclusion time of 10-40 s, and a charge valence state of 2⁺-8⁺.

The invention has the following advantageous effects.

The invention provides a preservation method for a urine sample by selecting PVDF filter plates for urine protein preservation, which can ensure stable urine protein quantity and quality within 1 year.

The invention provides a pretreatment method for a urine sample, which significantly increases the protein adsorption rate when reducing the pretreating time of the sample, so as to improve the effectiveness and accuracy of urine proteomics analysis.

The invention also provides an automatic treatment system and an automatic sample treatment method. The treatment system greatly reduces the labor intensity of people, is beneficial to speed up the treatment efficiency of urine sample treatment, meets the needs of high-flux and automated pretreatment of the proteomics, and meets the reproducibility and flux of current clinical needs.

### BRIEF DESCRIPTION OF THE DRAWINGS

In order to more clearly describe the technical solutions in the embodiments of the invention, the drawings to be used in the embodiments will be briefly introduced below. The drawings in the following description are only some embodiments of the invention, and thus should not be deemed as limiting the scope of the invention. It will be apparent to those skilled in the art that other drawings may be obtained from the drawings without any creative efforts.

FIG. 1 is a flow chart of urine sample preservation and pretreatment;

FIG. 2 is a software operation interface for urine sample preservation and automated pretreatment of the proteomics;

FIG. 3 shows an internal structure of a workstation for urine sample preservation and automated pretreatment of the proteomics (1. thermostatic mixing shaker; 2. 200 μL pipette tip; 3. PCR plate; 4. 50 μL pipette tip; 5-PCR plate; 6-low temperature disk; 7. twelve-channel tank; 8. 0.5 mL 96-well plate; 9. PVDF filter plate);

FIG. 4 is a ranking graph of protein distribution of all samples;

FIG. 5 is a statistical graph of the results of protein identification of samples;

FIG. 6 is a statistical graph of identification of protein and peptide fragments thereof corresponding to different concentrations of urea, with black representing the number of proteins identified and gray representing the number of peptide fragments identified;

FIG. 7 is a statistical graph of identification of proteins and peptide fragments thereof corresponding to different preservation times, with black representing the number of proteins identified and gray representing the number of peptide fragments identified.

### DESCRIPTION OF THE EMBODIMENTS

In order to make the objectives, technical solutions, and advantages of the embodiments of the invention more apparent, the technical solutions in the embodiments of the invention will be described in detail in conjunction with the accompanying drawings in the embodiments of the present application. Where specific conditions are not specified in the embodiments, they are carried out according to conventional conditions or conditions suggested by the manufacturer. Where the reagents or instruments used are not specified by the manufacturer, they are conventional products commercially available.

The characteristics and performance of the invention are further described in detail in the following embodiments.

### Comparative Example 1

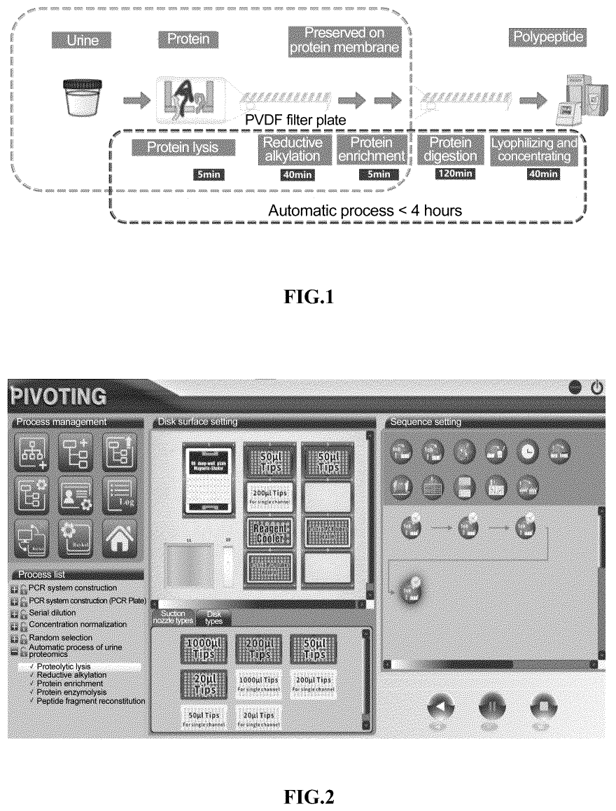

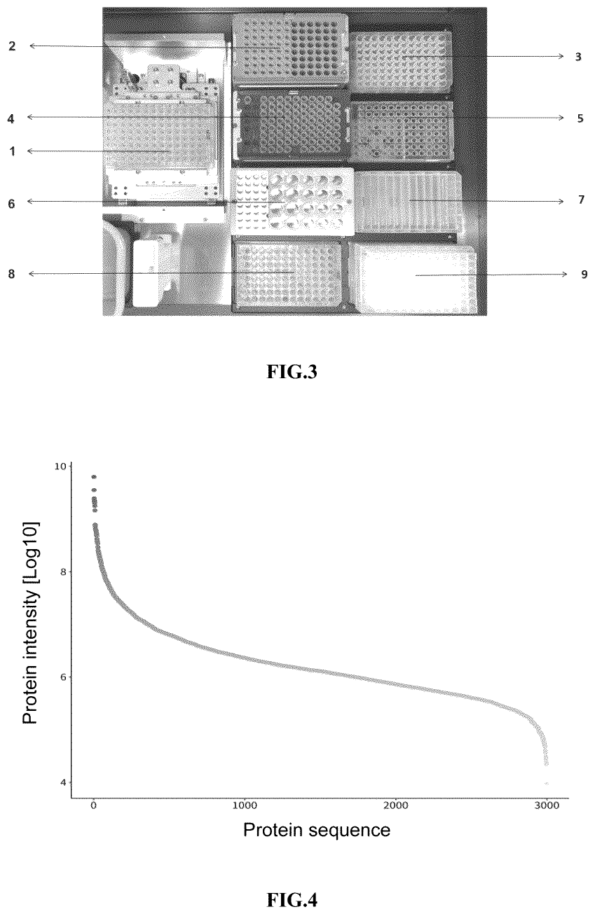

A pretreatment method (shown with reference to FIGS. 1 and 2) for urine samples (A, B, C, D urine samples all from healthy volunteers) includes the following steps.

### (1) Protein Lysis

Sample A: 100 μL of the same urine sample was added with 300 μL of 8M urea (diluent: 50 mM ammonium bicarbonate), with a final concentration of urea of 6M. The mixture was vortexed homogeneously to extract the protein.

### (2) Reductive Alkylation

Dithiothreitol was added to a product after protein lysis to a final concentration of 10 mM, and the reaction thereof was carried out at room temperature for 20 min. Iodoacetamide (alkylation) was added to the reduced product to a final concentration of 20 mM, and the reaction thereof was carried out in the dark for 20 min. An equal volume of dithiothreitol was added to the alkylated product to neutralize the excess iodoacetamide in the alkylation reaction.

### (3) Protein Enrichment

A PVDF filter plate activation was carried out by adding 200 μL 70% ethanol to the PVDF filter plate and centrifuging at 1000 g. PVDF filter plate equilibration was carried out by adding 200 μl of 6M urea (diluent: 50 Mm ammonium bicarbonate) to the PVDF filter plate, and centrifuging at 1000 g. The sample was then transferred to the PVDF filter plate and centrifuged at 1000 g. The sample was finally washed by adding 50 mM ammonium bicarbonate solution and centrifuged at 1000 g.

### (4) Protein Enzymolysis

100 μL of 50 mM ammonium bicarbonate solution and 1 μg of mixed trypsin and lysinase (LysC) were added. The mixture was shaken and incubated at 37° C. for 2 h, and centrifuged at 1000 g for 1 min after the completion of incubation to collect a filtrate. An additional 150 μL of 40% acetonitrile (containing 0.1% formic acid) was added to elute the peptide fragments and the eluent was combined.

### (5) Concentrating and Lyophilizing

The collected eluent was concentrated and lyophilized in a vacuum centrifugal concentrator.

### Comparative Example 2

The urea process is combined with a conventional pretreatment process as follows.

D samples were treated as follows. A 300 μL urine sample was added to 1500 μL of pre-cooled methanol according to the ratio of urine: methanol=1:5 (V/V). The mixture was vortexed for 20 s, allowed to stand at −20° C. for 1.5 h, and centrifuged for 10 min at 12000 g at 4° C. to discard a supernatant. The precipitate was washed once with 80% ethanol and dried in a concentrator. The sample was reconstituted with 50 μL of urea solution using the BSA method (Pierce™ BCA Protein Assay Kit, Brand: Thermo Fisher, Code: 23227) to determine the protein concentration. According to the protein concentration determined by the BSA method, 10 μg protein was added into a 96-well plate, and the mixture was made up to 50 μL of total volume by using 8 M urea. Dithiothreitol was added to a final concentration of 10 mM, and the reaction thereof was carried out at room temperature for 20 min. Iodoacetamide was added to a final concentration of 20 mM, and the reaction thereof was carried out for 20 min in the dark. An equal amount of dithiothreitol was added to neutralize the excess iodoacetamide. 1 μg of mixed trypsin and lysinase (LysC) was added. The mixture was incubated at 37° C. with shaking for 2 h, and 150 μL of 50 mM ammonium bicarbonate was added to dilute urea to below 2 M after the completion of incubation. The reaction was terminated by adding 20 μL of 10% trifluoroacetic acid to the reaction system, and followed by a desalting operation.

The desalting operation was as follows. 100 μL of methanol was added to the desalted plate and the mixture was centrifuged at 600 g for 1 min. 100 μL of 0.2% trifluoroacetic acid/80% acetonitrile was added and the mixture was centrifuged at 600 g for 1 min. 200 μL of 0.2% trifluoroacetic acid/water was added and the resultant was centrifuged at 600 g for 1 min. The sample was added and the mixture was centrifuged at 600 g for 1 min, which is repeated once. 200 μL of 0.2% trifluoroacetic acid/water was added and the mixture was centrifuged at 600 g for 1 min and rinsed. 100 μL of 0.2% trifluoroacetic acid/80% acetonitrile was added and the mixture was centrifuged at 600 g for 1 min for eluting. The filtrate was collected for concentration and lyophilization.

### Embodiment 1

A pretreatment method for a urine sample is substantially the same as Comparative Example 1, except for the different concentration of the protein lysate as follows.

Sample B: 200 μL of the same urine sample was added with 200 μL of 8M urea (diluent: 50 Mm ammonium bicarbonate), with the final concentration of urea of 4M. The mixture was vortexed homogeneously to extract the protein.

### Embodiment 2

A pretreatment method for a urine sample is substantially the same as Comparative Example 1, except for the different concentration of the protein lysate as follows.

Sample C: 300 μL of the same urine sample was added with 200 μL of 8M urea (diluent: 50 Mm ammonium bicarbonate), with the final concentration of urea of 3M. The mixture was vortexed homogeneously to extract the protein.

### Embodiment 3

A preservation method for a urine sample (shown with reference to FIG. 1) includes the following steps.

### (1) Protein Lysis

300 μL of the same urine sample (sample C) was taken (diluent: 50 Mm ammonium bicarbonate), with the final concentration of urea of 3 M. The mixture was vortexed homogeneously to extract the protein.

### (2) Reductive Alkylation

Dithiothreitol was added to a product after protein lysis to a final concentration of 10 mM, and the reaction thereof was carried out at room temperature for 20 min. Iodoacetamide (alkylation) was added to the reduced product to a final concentration of 20 mM, and the reaction thereof was carried out in the dark for 20 min. An equal volume of dithiothreitol was added to the alkylated product to neutralize the excess iodoacetamide in the alkylation reaction.

### (3) Protein Enrichment

A PVDF filter plate activation was carried out by adding 200 μL 70% ethanol to the PVDF filter plate and centrifuging at 1000 g. PVDF filter plate equilibration was carried out by adding 200 μl of 3M urea (diluent: 50 Mm ammonium bicarbonate) to the PVDF filter plate, and centrifuging at 1000 g. The sample was then transferred to the PVDF filter plate and centrifuged at 1000 g. The sample was finally washed by adding 50 mM ammonium bicarbonate solution and centrifuged at 1000 g for 1 min.

Urine protein samples stored in the PVDF filter plate were stored at −80° C. for 1 month.

### Embodiment 4

This embodiment provides a preservation method for an urine sample (as shown in FIG. 1), which differs from Embodiment 3 only in respect of the preservation period. In the embodiment, the urine protein samples stored in the PVDF filter plate were stored at −80° C. for 3 months.

### Embodiment 5

This embodiment provides a preservation method for a urine sample (as shown in FIG. 1), which differs from Embodiment 3 only in respect of the preservation period. In the embodiment, the urine protein samples stored in the PVDF filter plate were stored at −80° C. for 5 months.

### Embodiment 6

This embodiment provides a preservation method for a urine sample (as shown in FIG. 1), which differs from Embodiment 3 only in respect of the preservation period. In the embodiment, the urine protein samples stored in the PVDF filter plate were stored at −80° C. for 7 months.

### Embodiment 7

This embodiment provides a preservation method for a urine sample (as shown in FIG. 1), which differs from Embodiment 3 only in respect of the preservation period. In the embodiment, the urine protein samples stored in the PVDF filter plate were stored at −80° C. for 9 months.

### Embodiment 8

This embodiment provides a preservation method for a urine sample (as shown in FIG. 1), which differs from Embodiment 3 only in respect of the preservation period. In the embodiment, the urine protein samples stored in the PVDF filter plate were stored at −80° C. for 12 months.

### Embodiment 9

This embodiment provides an automatic treatment system for a urine sample. Specifically, the proteome pretreatment process of clinical urine samples is integrated into an automatic workstation. With reference to FIG. 3, the internal structure of the workstation includes a thermostatic mixing shaker 1, a 200 μL pipette tip 2, a PCR plate 3, a 50 μL pipette tip 4, a PCR plate 5, a low temperature disk 6, twelve-channel tank 7, a 0.5 mL 96-well plate 8 and a PVDF filter plate 9. The entire automation process can be divided into five parts: protein lysis, reductive alkylation, protein enrichment, protein digestion and concentration and lyophilization.

The automatic treatment system includes a urine sample storage unit, a treating fluid supply unit, a PVDF filter plate supply unit, a sample suction unit, a protein collection unit and an enzyme storage unit, wherein the urine sample storage unit, the treating fluid supply unit. The PVDF filter plate supply unit, the sample suction unit, the protein collection unit and the enzyme storage unit are electrically connected to a control terminal for automatic control.

The above treatment system further includes a lysis reaction vessel supply unit, a shaker, a concentrator and a PCR plate.

The treating fluid supply unit includes a lysate supply unit, a reducing agent supply unit, an alkylating agent supply unit, an alkylation reaction terminating agent supply unit, an eluent supply unit, an activating agent supply unit, and a reconstitution solvent supply unit. The treating fluid supply unit may be a twelve-channel tank, with a different reagent supply unit provided in each channel.

In this embodiment, the control terminal is a computer. The functions of automatic liquid supply, elution, sample loading, shaking and enrichment are realized by the control terminal.

Specifically, the automatic treatment system performs an automated urine sample treatment process as follows, and a more specific pretreatment experimental process is shown in Table 1.

Step 1-Protein lysis (as in Embodiment 2): A 300 μL of urine sample was transferred automatically and placed in a 0.5 mL 96-well plate and on a thermostatic mixing shaker at Position 1. Then, 200 μL of 8M urea (diluent: 50 Mm ammonium bicarbonate) was sucked and added into the 0.5 mL 96-well plate on the thermostatic mixing shaker at Position 1, respectively. The mixture was vortexed at a rotation speed of 1000 rpm to extract protein.

Step 2-reductive alkylation: 10 μL of 0.5 M dithiothreitol was sucked from Column 2 (A2) of twelve-channel tank at Position 7 and added into 0.5 mL 96-well plate on the thermostatic mixing shaker at Position 1 respectively for a final concentration of 10 mM. The mixture was vortexed homogeneously at the rotation speed of 1000 rpm, and reacted at room temperature for 20 min. 20 μl of 0.5 M iodoacetamide was sucked from Column 3 (A3) of the twelve-channel tank at Position 7 and added into the 0.5 mL 96-well plate on the thermostatic mixing shaker at Position 1 for a final concentration of 20 mM. The mixture was vortexed homogeneously at the rotation speed of 1000 rpm, and reacted in the dark for 20 min. Then, 10 μL of 0.5 M dithiothreitol was sucked from Column 2 (A2) of the twelve-channel tank at Position 7 and added into the 0.5 mL 96-well plate on the thermostatic mixing shaker at Position 1, respectively. The mixture was vortexed homogeneously at the rotation speed of 1000 rpm to neutralize excess iodoacetamide.

Step 3-Protein Enrichment: 200 μL of 70% ethanol was sucked from Column 4 (A4) of the twelve-channel tank at Position 7 and added into the PVDF-96 well plate (namely, a PVDF filter plate) at Position 9 respectively. The mixture was centrifuged at 1000 g to activate the PVDF filter plate. 200 μL of 3 M urea (diluent: 50 Mm ammonium bicarbonate) was sucked from Column 4 (A5) of the twelve-channel tank at Position 7 added into the PVDF filter plate at Position 9, respectively. The mixture was centrifuged at 1000 g for PVDF filter plate equilibration. Then, the sample after the completion of reductive alkylation in the 0.5 mL 96-well plate on the thermostatic mixing shaker at Position 1 was transferred into PVDF filter plate at Position 9, and the same was centrifuged at 1000 g. Finally, 100 μL of 50 mM ammonium bicarbonate solution was sucked from Column 6 (A6) of the twelve-channel tank at Position 7 to wash the sample, and the mixture was centrifuged at 1000 g.

Step 4-Protein digestion: 100 μL of 50 mM ammonium bicarbonate solution and 1 μg of mixed trypsin and lysinase (LysC) were sucked from Column 1 of the low-temperature disk at Position 6 (that is the enzyme storage unit) and respectively added into the PVDF filter plate at Position 9. Then, the PVDF filter plate at Position 9 was displaced to the thermostatic mixing shaker at Position 1 for shaking incubation at 37° C. at a rotation speed of 1000 rpm for 2 h. After the incubation is completed, the mixture was centrifugated at 1000 g for 1 min for collecting a peptide fragment filtrate. 150 μL of 40% acetonitrile (containing 0.1% formic acid) solvent was sucked from Column 7 (A7) of the twelve-channel tank at Position 7, and added into PVDF filter plate at Position 9 for elution. The mixture was centrifugated for 1 min at 1000 g, and all the eluents were combined.

Step 5-Concentrating and lyophilizing: the collected eluent was concentrated and lyophilized in a vacuum centrifugal concentrator.

According to the requirements of mass spectrometry detection, the automatic treatment system of the present application can be further used for a reconstitution operation. The concentrated and lyophilized peptide fragment sample was placed on the thermostatic mixing shaker at Position 1 of the workstation. 20 μL of 0.1% formic acid aqueous solvent was sucked from Column 8 (A8) of the twelve-channel tank at Position 7. The mixture was vortexed homogeneously at a rotation speed of 1000 rpm for 1 min to perform peptide reconstitution. After the completion of reconstitution, 15 μL of the supernatant was transferred from the 0.5 mL 96-well plate on the thermostatic mixing shaker at Position 1 into the PCR plate at Position 3, respectively, waiting for mass spectrometry detection and analysis to perform peptide fragment detection.

TABLE 1
Automatic treatment process

| Pretreatment | | | | |
|---|---|---|---|---|
| experimental | | | | Time |
| process | Automatic operation | Disk position | Reagent | consuming |
| Protein | 1. A robotic arm | 1. A 0.5 ml 96-well | 1. Urine | 1. 30 min |
| lysis | removed a 300 μL of | plate sample disk was | sample | 2. 1 min |
| | urine sample and | placed on the | 2. 8M urea | 3. 1 min |
| | placed the same in a | thermostatic mixing | solvent | |
| | 0.5 mL 96-well plate. | shaker at Position 1. | | |
| | 2. The robotic arm | 2. 8M Urea solvent | | |
| | removed 200 μL of | was placed in Column | | |
| | 8M urea (diluent: 50 | 1 (A1) of twelve- | | |
| | mM ammonium | channel tank (which is | | |
| | bicarbonate) and | the treating fluid | | |
| | added the same to | supply unit) at Position | | |
| | the 0.5 mL 96-well | 7. | | |
| | plate in the previous | | | |
| | step. | | | |
| | 3. The mixture was | | | |
| | vortexed | | | |
| | homogeneously at | | | |
| | the rotation speed of | | | |
| | 1000 rpm for extract | | | |
| | of the protein. | | | |
| Reductive | 1. 10 μL of 0.5M | 1.1 The 0.5 mL 96-well | 1. 0.5M | 1.1 1 min |
| alkylation | dithiothreitol was | plate sample disk was | dithiothreitol | 1.2 20 min |
| | transferred and | placed on the | 2. 0.5M | 2.1 1 min |
| | added into the 0.5 mL | thermostatic mixing | iodoacetamide | 2.2 20 min |
| | 96-well plate in the | shaker at Position 1. | 3. 0.5M | 3. 1 min |
| | previous step by the | 1.2 0.5M dithiothreitol | dithiothreitol | |
| | robotic arm. The | was placed in Column | | |
| | mixture was vortexed | 2 (A2) of the twelve- | | |
| | homogeneously at | channel tank (namely, | | |
| | the rotation speed of | the treating fluid | | |
| | 1000 rpm, and | supply unit) at Position | | |
| | reacted at room | 7; | | |
| | temperature for 20 | 2. 0.5M | | |
| | min; | iodoacetamide was | | |
| | 2. 20 μL of 0.5M | placed in Column 3 | | |
| | iodoacetamide was | (A3) of the twelve- | | |
| | transferred into the | channel tank (which is | | |
| | 0.5 mL 96-well plate | the treating fluid | | |
| | in the previous step | supply unit) at Position | | |
| | by robotic arm. The | 7. | | |
| | mixture was vortexed | 3. 0.5M dithiothreitol | | |
| | homogeneously at | was placed in Column | | |
| | the rotation speed of | 2 (A2) of the twelve- | | |
| | 1000 rpm, and | channel tank (which is | | |
| | reacted at room | the treating fluid | | |
| | temperature in the | supply unit) at Position | | |
| | dark for 20 min; | 7. | | |
| | 3. 10 μL of 0.5M | | | |
| | dithiothreitol was | | | |
| | transferred and | | | |
| | added into the 0.5 mL | | | |
| | 96-well plate in the | | | |
| | previous step with | | | |
| | the robotic arm. The | | | |
| | mixture was vortexed | | | |
| | homogeneously at | | | |
| | the rotation speed of | | | |
| | 1000 rpm. | | | |
| Protein | 1. The robotic arm | 1.1 70% ethanol was | 1.1 70% | 1. 1 min |
| enrichment | transferred 200 μL of | placed in Column 4 | Ethanol | 2. 1 min |
| | 70% ethanol and | (A4) of the twelve- | 2. 3M Urea | 3. 1 min |
| | respectively added | channel tank at | (diluent: 50 | 4. 1 min |
| | the same into a | Position 7. | mM | |
| | PVDF-96 well plate | 1.2 PVDF filter plates | ammonium | |
| | (namely, a PVDF | were placed at | bicarbonate) | |
| | filter plate) to activate | Position 9. | 4. 50 mM | |
| | the PVDF filter plate. | 2. 3M Urea (diluent: 50 | ammonium | |
| | 2. The robotic arm | Mm ammonium | bicarbonate | |
| | transferred 200 μL of | bicarbonate) was | solution | |
| | 3M urea (diluent: 50 | placed in Column 4 | | |
| | mM ammonium | (A5) of the twelve- | | |
| | bicarbonate) and | channel tank at | | |
| | respectively added | Position 7. | | |
| | the same into the | 3. The 0.5 mL 96-well | | |
| | PVDF filter plate | plate sample disk was | | |
| | respectively for | placed on the | | |
| | PVDF filter plate | thermostatic mixing | | |
| | equilibration. | shaker at Position 1. | | |
| | 3. Then, the sample after | 4. 50 Mm ammonium | | |
| | the reductive | bicarbonate solution | | |
| | alkylation in 0.5 mL | was placed in Column | | |
| | 96-well plate was | 6 (A6) of the 12- | | |
| | transferred into the | channel tank at | | |
| | PVDF filter plate to | Position 7. | | |
| | perform | | | |
| | centrifugation. | | | |
| | 4. The robotic arm | | | |
| | transferred 100 μL 50 | | | |
| | mM ammonium | | | |
| | bicarbonate solution | | | |
| | to wash the sample, | | | |
| | and the mixture is | | | |
| | then performed with | | | |
| | centrifugation. | | | |
| Protein | 1.1 The robotic arm | 1.1 Trypsin and | 1.1 Trypsin | 1.1 1 min |
| digestion | transferred 100 μL of 50 | lysinase (LysC) were | and lysinase | 1.2 1 min |
| | mM ammonium | placed in Column 1 of | (LysC) | 2. 2 h |
| | bicarbonate solution and | the low temperature | 3. 40% | 3. 1 min |
| | 1 μg of mixed trypsin and | disk (namely, the | acetonitrile | |
| | lysinase (LysC). | enzyme storage unit) | (containing | |
| | 1.2 The mixture was | at Position 6. | 0.1% formic | |
| | added into the PVDF | 1.2 The PVDF filter | acid) solvent | |
| | filter plate respectively. | plate was placed at | | |
| | 2. Then, the PVDF filter | Position 9. | | |
| | plate was displaced to a | 2. The thermostatic | | |
| | thermostatic mixing | mixing shaker was | | |
| | shaker for shaking | placed at Position 1. | | |
| | incubation at 37° C. at a | 3. A solvent of 40% | | |
| | rotation speed of 1000 | acetonitrile (containing | | |
| | rpm for 2 h. | 0.1% formic acid) was | | |
| | 3. After the completion of | placed in Column 7 | | |
| | the incubation, the | (A7) of the 12-channel | | |
| | peptide fragment filtrate | tank (namely, the | | |
| | was collected by | treating fluid supply | | |
| | centrifugation. The | unit) at Position 7. | | |
| | robotic arm transferred | | | |
| | 150 μL of 40% | | | |
| | acetonitrile (containing | | | |
| | 0.1% formic acid) and | | | |
| | added the same to the | | | |
| | PVDF filter plate for | | | |
| | centrifugal operation. | | | |
| | Finally, all the peptide | | | |
| | fragment filtrates were | | | |
| | combined. | | | |
| Concentrating | 1. The combined filtrates | 1. The 0.5 ml 96-well | 2.1 Aqueous | 1. 1 min |
| and | were concentrated and | plate sample disk was | solution | 2.1 1 min |
| lyophilizing | lyophilized in a vacuum | placed on the | containing | 2.2 1 min |
| | centrifugal concentrator. | thermostatic mixing | 0.1% formic | 3. 1 min |
| | The peptide fragment | shaker at Position 1. | acid | |
| | sample was placed on a | 2.1 The aqueous | | |
| | thermostatic mixing | solution containing | | |
| | shaker at Position 1 of | 0.1% formic acid was | | |
| | the workstation; | placed in Column 8 | | |
| | 2.1 20 μL of aqueous | (A8) of a twelve- | | |
| | solution containing 0.1% | channel tank (namely, | | |
| | formic acid was sucked | the treating fluid | | |
| | from Column 8 (A8) of | supply unit) at Position | | |
| | the twelve-channel tank | 7. | | |
| | at Position 7, and added | 2.2 The 0.5 mL 96-well | | |
| | into the sample at | plate sample disk was | | |
| | Position 1. | placed on the | | |
| | 2.2 The mixture was | thermostatic mixing | | |
| | vortexed | shaker at Position 1 for | | |
| | homogeneously at 1000 | vortex mixing; | | |
| | rpm for 1 min to perform | 3. The peptide | | |
| | peptide reconstitution. | fragment sample after | | |
| | 3. 15 μL of supernatant | reconstitution was | | |
| | was transferred from the | transferred to the PCR | | |
| | 0.5 mL 96-well plate at | plate at Position 3. | | |
| | Position 1 into the PCR | | | |
| | plate at Position 3 | | | |
| | respectively. | | | |

The chromatographic and mass spectrometric detection parameters in this embodiment are as follows.

On-line detection of liquid phase parameters: a mobile phase A is set as an aqueous solution containing 0.1% formic acid and a mobile phase B as 80% acetonitrile containing 0.1% formic acid, with gradient elution conditions as shown in Table 2. The chromatographic column is Acclaim™ PepMap™ 100 C₁₈ (Thermo Fisher, 0.075 mm, 20 mm), with the column temperature of 55° C.

TABLE 2
Gradient Elution Table

| Time | Mobile Phase | Mobile Phase | Flow Rate |
|---|---|---|---|
| (min) | A | B | (nL/min) |
| 0 | 99 | 1 | 300 |
| 1 | 99 | 1 | 300 |
| 3 | 94 | 6 | 300 |
| 6 | 92 | 8 | 300 |
| 23 | 70 | 30 | 300 |
| 27 | 1 | 99 | 300 |
| 30 | 1 | 99 | 300 |

On-line detection of mass spectrometry parameters. A mass spectrum full scan resolution is 60,000@m/z 200. AGC is 3E6. The maximum ion injection time is 100 ms. The scan range is m/z 200-2000. The normalized collision energy is 27%. The secondary mass spectrum scan resolution is 15,000@m/z 200. The scan range is m/z 200-2000. AGC is 1E6. The maximum ion injection time is 50 ms. The dynamic exclusion time is 40 s. The charge valence state is 2⁺-8⁺.

After the completion of sample detection, the quantitative intensity of all samples was statistically analyzed (FIG. 4). The results showed that the quantitative results of all samples spanned 6 orders of magnitude. The peptide fragment intensity may be detected from the lower 4 to the higher 10 of intensity (Log 10), and the intensity distribution was stable, indicating that the data coverage was wide and may be used for later analysis.

As shown in FIG. 5, most of the identified proteins in the samples were distributed between 1000 and 2500, and the number of identified proteins was relatively stable.

### Experimental Example 1

The A, B, C, D samples in Embodiments 1-2 and Comparative Examples 1-2 were all reconstituted with an aqueous solution containing 0.1% formic to the similar concentration of 1 μg/μL for peptide fragment detection by the mass spectrometric detection analysis.

On-line detection of liquid phase parameters: the mobile phase A is the aqueous solution containing 0.1% formic acid and the mobile phase B is 80% acetonitrile containing 0.1% formic acid. The gradient elution conditions are as shown in Table 2. The chromatographic column is Acclaim™ PepMap™ 100 C₁₈ (Thermo Fisher, 0.075 mm, 20 mm), with the column temperature of 55° C. On-line detection of mass spectrometry parameters. A mass spectrum full scan resolution is 60,000@m/z 200. AGC is 3E6. The maximum ion injection time is 100 ms. The scan range is m/z 200-2000. The normalized collision energy is 27%. The secondary mass spectrum scan resolution is 15,000@m/z 200. The scan range is m/z 200-2000. AGC is 1E6. The maximum ion injection time is 50 ms. The dynamic exclusion time is 40 s. The charge valence state is 2⁺-8⁺.

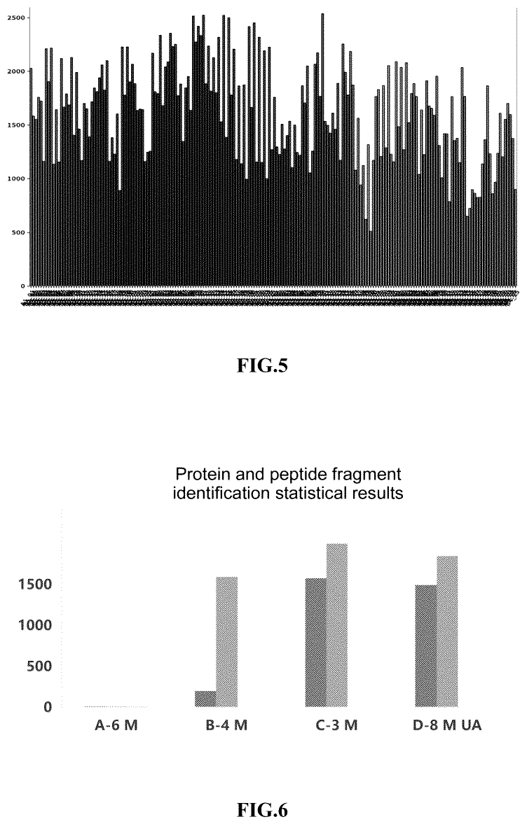

As shown in FIG. 6, compared with a sample D (the urea method in combination with the traditional pretreatment operation process), the mass spectrum of sample A (the urea final concentration: 6M) has apparently no peptide fragment to be detected. The mass spectrum of sample B (the urea concentration: 4M) has slightly more peptide fragment information than that of the sample A. A sample C (the urea concentration 3 M) has more peaks to be detected, and the detection tendency is consistent with the traditional pretreatment method (sample D). The spectrum is subjected to library search, and the detected peptide fragment information is subjected to protein identification. The statistical results of identification are shown in FIG. 6. Therefore, the PVDF filter plate combined with the urea with the final concentration of 3M is used for pre-proteomic treatment of urine samples, which not only overcomes the defects of traditional FASP method that could not be used for high-flux sample treatment during the protein sample pretreatment, but also improves the detection results of protein in the urine samples.

### Experimental Example 2

This experimental example demonstrates the stability of samples obtained by the urine sample preservation method provided by the invention.

After the preservation was expired, dithiothreitol was added to the samples in 0 month (i.e., performing subsequent pretreatment operation immediately after protein lysis), 1 month (i.e., after the completion of protein enrichment in Example 3 and the preservation of urine protein in the PVDF filter plate for 1 month), 3 months (i.e., after the completion of protein enrichment in Example 4 and the preservation of urine protein in the PVDF filter plate for 3 months), 5 months (i.e., after the completion of protein enrichment in Example 5 and the preservation of urine protein in the PVDF filter plate for 5 months), 7 months (i.e., after the completion of protein enrichment in Example 6 and the preservation of urine protein in the PVDF filter plate for 7 months), 9 months (i.e., after the completion of protein enrichment in Example 7 and the preservation of urine protein in the PVDF filter plate for 9 months) and 12 months (i.e., after the completion of protein enrichment in Example 8 and the preservation of urine protein in the PVDF filter plate for 12 months), respectively, for a final concentration of 10 mM, and the reaction thereof was carried out at room temperature for 20 min. Iodoacetamide was added to a final concentration of 20 mM and reacted for 20 min in the dark. An equal amount of dithiothreitol was added to neutralize the excess iodoacetamide. 200 μl of 70% ethanol was added and the mixture was centrifuged at 1000 g for PVDF filter plate activation. 200 μL 3M urea (diluent: 50 mM ammonium bicarbonate) was added. The mixture was centrifuged at 1000 g for PVDF filter plate equilibration. The sample was then transferred to the PVDF filter plate and centrifuged at 1000 g. Finally, 50 mM ammonium bicarbonate solution was added to wash the sample, and the mixture was centrifuged at 1000 g. 100 μL of 50 mM ammonium bicarbonate solution and 1 μg of mixed trypsin and lysinase (LysC) were added. The mixture was incubated at 37° C. with shaking for 2 h. After the completion of incubation, the mixture was centrifuged at 1000 g for 1 min for collecting the filtrate. Then 150 μL of 40% acetonitrile (containing 0.1% formic acid) was added to elute the peptide fragments. The filtrates were combined, concentrated and lyophilized, redissolved to 1 μg/μL with aqueous solution containing 0.1% formic acid, and analyzed by the mass spectrometry for peptide fragment detection.

On-line detection of liquid phase parameters: the mobile phase A is set to the aqueous solution containing 0.1% formic acid and the mobile phase B to 80% acetonitrile containing 0.1% formic acid. The gradient elution program is shown in Table 2. The chromatographic column is Acclaim™ PepMap™ 100 C₁₈ (Thermo Fisher, 0.075 mm, 20 mm), with the column temperature of 55° C.

On-line detection of mass spectrometry parameters. A mass spectrum full scan resolution is 60,000@m/z 200. AGC is 3E6. The maximum ion injection time is 100 ms. The scan range is m/z 200-2000. The normalized collision energy is 27%. The secondary mass spectrum scan resolution is 15,000@m/z 200. The scan range is m/z 200-2000. AGC is 1E6. The maximum ion injection time is 50 ms. The dynamic exclusion time is 40 s. The charge valence state is 2⁺-8⁺.

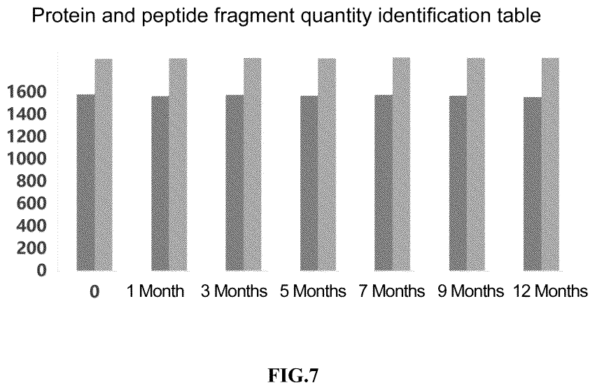

The statistical results of protein detection in this experiment are shown in FIG. 7. From the statistical results of protein detection, it is found that when the urine protein is stored in the PVDF filter plate for 12 months, the number of protein and peptide fragments detected on line is relatively stable, and is maintained within the range of 1500 protein and 12000 peptide fragments, with no significant fluctuation. Therefore, the invention provides a preservation method for an urine sample which is reliable and can ensure stable protein quantity and quality within 1 year.

The above mentioned are merely preferred embodiments of the invention and not intended to limit the invention. There are various modifications and changes in this invention for those skilled in the art. Any modifications, equivalents, improvements, etc. within the spirit and principles of this invention are intended to be included within the scope of this invention.

1. A preservation method for a urine sample, comprising:
subjecting the urine sample after protein lysis to a reductive alkylation treatment,
and then followed by a protein enrichment;
wherein the protein enrichment is performed on the sample after the reductive alkylation treatment using a PVDF filter plate for protein enrichment;
the mixture volume ratio of a lysate used for protein lysis to the urine sample to be lysed is 1:0.1-9.

2. The preservation method for the urine sample according to claim 1, characterized in that the lysate is at least one selected from the group consisting of urea, thiourea, guanidine hydrochloride, tris (hydroxymethyl) aminomethane-hydrochloride, phenylmethylsulfonyl fluoride, sodium dodecyl sulfate, sodium deoxycholate and 3-[3-(cholamidopropyl) dimethylammonio]-1-propanesulfonate;
preferably, the lysate is selected from urea and the final concentration of the urea in the urine sample to be lysed is 1M-5M;
preferably, a diluent for the protein lysate is at least one selected from the group consisting of ammonium bicarbonate, tris (hydroxymethyl) aminomethane-hydrochloride solution, phosphate solution.

3. The preservation method for the urine sample according to claim 1, further comprising, before the protein enrichment, activating the PVDF filter plate, equilibrating with the lysate after the activation, and thereafter transferring the sample after the reductive alkylation treatment to the equilibrated PVDF filter plate for protein enrichment;
preferably, an activating agent for the activation is an alcohol.

4. A pretreatment method for a urine sample, comprising: subjecting the urine sample after protein lysis to a reductive alkylation treatment, then protein enrichment, thereafter enzymolysis, and concentrating and lyophilizing;
wherein the protein enrichment is performed on the sample after the reductive alkylation treatment using a PVDF filter plate for protein enrichment;
the mixture volume ratio of a lysate used for protein lysis to the urine sample to be lysed is 1:0.1:9.

5. The pretreatment method for the urine sample according to claim 4, characterized in that it also comprises collecting a filtrate from the PVDF filter plate after the enzymolysis;
preferably, the enzymes for the enzymolysis are trypsin and lysinase;
preferably, the time for enzymolysis is 1-18 h;
preferably, the protein enrichment comprises adding the sample after the reductive alkylation treatment to the PVDF filter plate, and after centrifugation, washing the centrifuged sample with an eluent.

6. The pretreatment method for the urine sample according to claim 4, characterized in that the lysate is at least one selected from the group consisting of urea, thiourea, guanidine hydrochloride, tris (hydroxymethyl) aminomethane-hydrochloride, phenylmethylsulfonyl fluoride, sodium dodecyl sulfate, sodium deoxycholate and 3-[3-(cholamidopropyl)dimethylammonio]-1-propanesulfonate;
preferably, the lysate is selected from urea and the final concentration of the urea in the urine sample to be lysed is 1M-5M.

7. An automatic treatment system for a urine sample, comprising a urine sample storage unit, a treating fluid supply unit, a PVDF filter plate supply unit, a sample suction unit, a protein collection unit and an enzyme storage unit, wherein the urine sample storage unit, the treating fluid supply unit, the PVDF filter plate supply unit, the sample suction unit, the protein collection unit and the enzyme storage unit are electrically connected to a control terminal for automatic control.

8. The automatic treatment system for the urine sample according to claim 7, characterized in that the automatic treatment system further comprises a lysis reaction vessel supply unit, a shaker, a concentrator, and a PCR plate;
wherein the treating fluid supply unit includes a lysate supply unit, a reducing agent supply unit, an alkylating agent supply unit, an alkylation reaction terminating agent supply unit, an activating agent supply unit, an eluent supply unit, and a reconstitution solvent supply unit.

9. A treatment method for a urine sample by using the automatic treatment system for the urine sample according to claim 7, comprising:
(1) protein lysis: taking a urine sample to be tested from a urine sample storage unit into a lysis reaction vessel by using a sample suction unit, and sucking a lysate from a treating fluid supply unit into the lysis reaction vessel via the sample suction unit to perform the protein lysis;
(2) reductive alkylation: sucking a reducing agent from the treating fluid supply unit into the lysis reaction vessel via the sample suction unit to perform a reduction reaction, sucking an alkylating agent from the treating fluid supply unit into the lysis reaction vessel via the sample suction unit to perform an alkylation reaction, and then sucking an alkylation reaction terminating agent from the treating fluid supply unit via the sample suction unit to terminate the alkylation reaction;
(3) protein enrichment: activating the PVDF filter plate by sucking an activating agent from the treating fluid supply unit into the PVDF filter plate via the sample suction unit, equilibrating the PVDF filter plate by sucking the lysate from the treating fluid supply unit into the PVDF filter plate via the sample suction unit, then adding a product after the reductive alkylation treatment to the PVDF filter plate via the sample suction unit, and centrifuging;
(4) proteolysis: sucking an enzyme reaction solution from an enzyme storage unit into the PVDF filter plate via the sample suction unit to perform an enzymolysis reaction, then sucking an eluent from the treating fluid supply unit into the PVDF filter plate via the sample suction unit to elute an enzymolysis reaction product, and then combining the eluent; and
(5) concentrating and lyophilizing: concentrating and lyophilizing the eluent.

10. A method of mass spectrometric detection for a urine sample, which is directed for the purpose of non-diagnosis of disease, characterized by comprising: pre-treating the urine sample by using the method of claim 9, and then performing peptide fragment detection by using a mass spectrometer;
setting a mobile phase A as an aqueous solution containing 0.05-0.2% formic acid and a mobile phase B as 80% acetonitrile containing 0.05-0.2% formic acid for gradient elution, with a flow rate of 200-300 nl/min and a column temperature of 30-55° C.;
preferably, the gradient elution has procedures of 1-6 min, 1%-8% B, 6-30 min, 8-99% B;
setting mass spectrometry parameters, including a mass spectrum full scan resolution of 240,000, 120,000, 70,000, 60,000, 45,000, 30,000, 17,500, 15,000 or 7,500@m/z 200, AGC of 1E5-3E6, maximum ion sample injection time of 10-100 ms, a scan range of m/z 200-2000, normalized collision energy of 15-27%; a secondary mass spectrum scan resolution of 240,000, 120,000, 70,000, 60,000, 45,000, 30,000, 17,500, 15,000 or 7,500@m/z 200, a scanning range of m/z 200-2000, an AGC of 1E5-1E6, maximum ion injection time of 10-100 ms, dynamic exclusion time of 10-40 s, and a charge valence state of 2⁺-8⁺.
